(19) United States
(12) Patent Application Publication
(10) Pub. No.: US 2006/0100456 A1
(43) Pub. Date: May 11, 2006

(54) Diastereoselective method of preparing olefins by means of the horner-wadsworthemmons reaction, comprising the addition of a tris-(polyoxaalkyl)-amine sequestering agent

(76) Inventors: Fracois Touchard, Villeurbanne (FR); Olivier Buisine, Lyon (FR); Frederic Leguyader, Lyon (FR); Thierry Schlama, Dardilly (FR); Franco Manfre, Ecully (FR)

Correspondence Address: Jean-Louis Seugnet;Rhodia Inc, Intellectual Property Dept, 259 Prospect Plains Road CN-7500, Cranbury, NJ, 08512-7500, US

(21) Appl. No.: 10/528,715

(22) PCT Filed: Sep. 22, 2003
(86) PCT No.: PCT/FR03/02781

§ 371 Date: Oct. 3, 2005

(30) Foreign Application Priority Data
Sep. 23, 2002 (FR) 02/11731

(51) Int. Cl. C07F 9/22; C07C 315/04; C07C 253/30; C07C 231/12

(52) U.S. Cl. 558/357; 562/598; 564/123; 562/8; 562/30; 568/27

(57) ABSTRACT

The invention relates to a process for the diastereoselective preparation of olefins via the Homer-Wadsworth-Emmons reaction, which consists in reacting at low temperature a phosphonate with a carbonyl derivative in the presence of a base in a suitable solvent, characterized in that a tris(polyoxaalkyl)amine sequestering reagent of formula (I): N—[CHR₁—CHR₂—O—(CHR₃—CHR₄—O)ₙ—R₅]₃ (I), wherein: n is an integer between 0 and 10; R₁, R₂, R₃ and R₄ may be identical or different, and represent a hydrogen atom or an alkyl radical containing from 1 to 4 carbon atoms; R₅ represents a hydrogen atom, an alkyl or cycloalkyl radical containing up to 12 carbon atoms, a phenyl radical or a radical of formula —CμH2μ-Φ, or CₘH₂ₘ₊₁-Φ-, with m being an integer between 1 and 12 and Φ being a phenyl radical; is added in an amount that is sufficient to increase the diastereoselectivity of the olefin.

The present invention relates to a process for the diastereoselective preparation of olefins via the Horner-Wadsworth-Emmons reaction, which consists in reacting at low temperature a phosphonate with a carbonyl derivative in the presence of a base, in a suitable solvent.

The reaction involved is as follows:

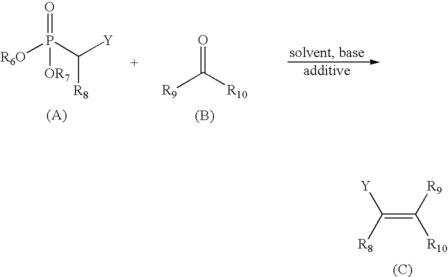

The carbonyl compound (B) may be an aldehyde or a ketone, with the condition that R₉ takes precedence over R₁₀ according to the Cahn Ingold and Prelog rules. These rules are described, for example, in the book entitled “Advanced Organic Chemistry” Reactions, Mechanisms, and Structure, third edition, Jerry March, John Wiley & sons, 1985, the content of pages 96 to 112 of which is incorporated by reference.

It is known practice from Tetrahedron Letters, Vol. 24, No. 41, pages 4405-4408, 1983 to use in this reaction five equivalents of a macrocyclic complexing agent of particular crown ether type, 18-crown-6 (18C6) to improve the diastereoselectivity of the olefin (C) obtained.

However, this crown ether has the drawback of being expensive, toxic and harmful to the environment. There was a need to find another means for improving the diastereoselectivity of the olefin obtained without using this crown ether.

The Applicant has just discovered, unexpectedly, that the use of a tris(polyoxaalkyl)-amine makes it possible to improve the diastereoselectivity in the Homer-Wadsworth-Emmons reaction to levels comparable to those obtained with 18-crown-6.

Thus, one subject of the present invention is a process for the diastereoselective preparation of olefins (C) via the Homer-Wadsworth-Emmons reaction, which consists in reacting at low temperature a phosphonate (A) with a carbonyl derivative (B) in the presence of a base, in a suitable solvent, 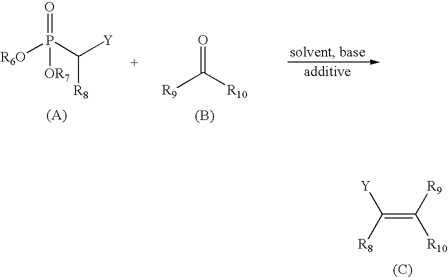
in which the compounds (A) (B) and (C) are such that:

Y represents an electron-withdrawing group known to those skilled in the art and chosen so as not to disrupt the Homer-Wadsworth-Emmons reaction. Among these groups, mention may be made especially of:
—CO₂R,
—CN,
—C(O)R,
—S(O)R,
—S(O)₂R,
—C(O)NRR′,
—N═CRR′,
—P(O)OROR′,
with R and R′ as defined below,

R₆ and R₇, taken independently, may be identical or different and represent:
a saturated or unsaturated, linear or branched aliphatic radical containing from 1 to 24 carbon atoms, optionally substituted with hetero atoms;
a saturated, unsaturated or aromatic, monocyclic or polycyclic cycloaliphatic radical containing from 4 to 24 carbon atoms, optionally substituted with hetero atoms;
a saturated or unsaturated, linear or branched aliphatic radical bearing a cyclic substituent optionally substituted with hetero atoms in the aliphatic part and/or the cyclic part;

R₁₀, R and R′, taken independently, may be identical or different and represent:
a hydrogen atom;
a saturated or unsaturated, linear or branched aliphatic radical containing from 1 to 24 carbon atoms, optionally substituted with hetero atoms;
a saturated, unsaturated or aromatic, monocyclic or polycyclic cycloaliphatic radical containing from 4 to 24 carbon atoms, optionally substituted with hetero atoms;
a saturated or unsaturated, linear or branched aliphatic radical bearing a cyclic substituent optionally substituted with hetero atoms in the aliphatic part and/or the cyclic part;

R₆, R₇, R and R′ may also be taken together to form a saturated, unsaturated or aromatic ring optionally comprising hetero atoms;

R₈ represents a radical chosen from:
—R,
a halogen atom,
—OR,
—NRR′,
with R and R′ as defined above,

R₉ represents a radical chosen from:
a saturated or unsaturated, linear or branched aliphatic radical containing from 1 to 24 carbon atoms, optionally substituted with hetero atoms;
a saturated, unsaturated or aromatic, monocyclic or polycyclic cycloaliphatic radical containing from 4 to 24 carbon atoms, optionally substituted with hetero atoms; the hetero atoms also possibly being present in the cyclic part;
a saturated or unsaturated, linear or branched aliphatic radical bearing a cyclic substituent optionally substituted with hetero atoms in the aliphatic part and/or the cyclic part;
   with the condition that R₉ takes precedence over R₁₀ according to the Cahn Ingold and Prelog rules,
   characterized in that a tris(polyoxaalkyl)amine sequestering agent of formula (I):
   N—[CHR₁—CHR₂—O—(CHR₃—CHR₄—O)ₙ—R₅]₃ (I)
   in which:

n is an integer between 0 and 10;

R₁, R₂, R₃ and R₄ may be identical or different and represent a hydrogen atom or an alkyl radical containing from 1 to 4 carbon atoms;

R₅ represents a hydrogen atom, an alkyl or cycloalkyl radical containing up to 12 carbon atoms, a phenyl radical or a radical of formula —CₘH₂ₘΦ, or CₘH₂ₘ₊₁-Φ-, with m being an integer between 1 and 12 and Φ being a phenyl radical;

is added to the reaction medium in an amount that is effective to increase the diastereo-selectivity of the olefins (C).

Preferably, the tris(polyoxaalkyl)amine sequestering agent used is one of formula (I) in which:

R₁, R₂, R₃ and R₄ may be identical or different and represent a hydrogen atom or a methyl radical;

n is an integer between 0 and 3;

R₅ represents a hydrogen atom or an alkyl radical containing from 1 to 4 carbon atoms.

Even more preferably, the tris(polyoxaalkyl)amine sequestering agent used is one of formula (I) in which:

R₁, R₂, R₃ and R₄ represent a hydrogen atom;

n is 1;

R₅ represents a methyl radical.

The tris(polyoxaalkyl)amine sequestering agent of formula (I) may be used in an amount ranging from 0.05 to 10 equivalents per 1 equivalent of phosphonate, one equivalent of aldehyde and one equivalent of base.

Preferably, the amount of tris(polyoxaalkyl)amine sequestering agent of formula (I) used is from 0.1 to 5 equivalents per 1 equivalent of phosphonate, one equivalent of aldehyde and one equivalent of base.

Even more preferably, the amount of tris(polyoxaalkyl)amine sequestering agent of formula (I) used is 1 equivalent per 1 equivalent of phosphonate, one equivalent of aldehyde and one equivalent of base, the whole being dissolved in a solvent.

The phosphonate used for the reaction may be chosen from phosphonates of formula (A): in which

Y represents CO₂R, with R representing a hydrogen atom or a linear, branched or cyclic, saturated or unsaturated alkyl radical containing from 1 to 12 carbon atoms,

R₆ and R₇ represent a —CH₂CF₃ radical, and R₈ represents a hydrogen atom.

Preferably, a phosphonate of formula (A) is used in which:

Y represents a radical CO₂R, and R represents a methyl radical;

R₆ and R₇ represents a —CH₂CF₃ radical; and R₈ represents a hydrogen atom.

The carbonyl derivative (B) used for the reaction may be an aldehyde or a ketone. The substituents R₉ and R₁₀ are, of course, chosen so as not to disrupt the Homer-Wadsworth-Emmons reaction. One condition according to the Cahn, Ingold and Prelog rule has been set, so as to define the selectivity of the olefin (C). The Cahn Ingold and Prelog rule is described, for example, in the book entitled “Advanced Organic Chemistry” Reactions, Mechanisms, and Structure, third edition, Jerry March, John Wiley & sons, 1985, the content of pages 96 to 112 of which is incorporated by reference.

The carbonyl derivative (B) is preferably chosen from aldehydes, which corresponds to R₁₀ representing a hydrogen atom. The aldehydes used may be, depending on the nature of the radical R₉, aliphatic, and optionally comprise ethylenic unsaturations, or they may be aromatic. In the case where the aldehydes used are aromatic, they may comprise optional substitutions with electron-donating or electron-withdrawing groups. Electron-donating groups that may be mentioned include C1-C6 alkyl, C1-C6 alkoxy and phenyl groups, where appropriate substituted with an alkyl or alkoxy group as defined above.

For the purposes of the present invention, the term “electron-withdrawing group” means a groups as defined by H. C. Brown in the book entitled “Advanced Organic Chemistry” Reactions, Mechanisms, and Structure, third edition, Jerry March, John Wiley & sons, 1985, the content of pages 243 and 244 of which is incorporated by reference.

Representative electron-withdrawing groups that may especially be mentioned include:
a halogen atom
a group SO₂R with R as defined above
a CN or NO₂ group.

An aromatic aldehyde is preferably used.

Among the aliphatic aldehydes that may be mentioned are cyclohexanecarboxaldehyde (R₉ is a cyclohexyl radical) or an aliphatic aldehyde in which R₉ is n-C₇H₁₅. The aliphatic aldehyde in which R₉ is a cyclohexyl radical is preferably used.

Among the aromatic aldehydes that may be mentioned are benzaldehyde (R₉ represents a phenyl radical) or an aldehyde characterized in that the radical R₉ used is aromatic and optionally comprises one or more substitutions with alkoxy groups containing from 1 to 6 carbon atoms or halogen atoms.

Examples that may also be mentioned include the aldehydes listed in Table VII.

Thus, the aromatic aldehyde may comprise hetero atoms in the aromatic ring.

The aromatic aldehyde may also comprise substitutions with CF₃ groups.

The base is chosen from:

amides of the type MNR″R′″ with M being an alkali metal such as lithium, sodium or potassium, and R″, R′″ being chosen from alkyl radicals or radicals of alkylsilane type, such as the potassium salt of hexamethyldisilazane (KHMDS),

alkoxides of the type MOR″ with M being an alkali metal such as lithium, sodium or potassium, and R″ being chosen from alkyl radicals, such as potassium tert-butoxide (KOtBu),

hydrides of the type MH with M being an alkali metal such as lithium, sodium or potassium,

carbonates of the type M₂CO₃ or MCO₃, with M being an alkali metal such as lithium sodium, potassium or cesium, or an alkaline-earth metal such as calcium or barium,

alkali metal or alkaline-earth metal hydroxides such as LiOH, NaOH, KOH, CsOH, Mg(OH)₂, Ca(OH)₂ and Ba(OH)₂,

organic bases, for instance 1,8-diazabicyclo[5.4.0]undec-7-ene (DBU), 1,1,3,3-tetra-methylguanidine (TMG) or 1,4-diazabicyclo[2.2.2]octane (DABCO) in combination with alkali metal or alkaline-earth metal halides.

The potassium salt of hexamethyldisilazane (KHMDS) or potassium tert-butoxide (KOtBu) is preferably used.

The solvent used is an organic solvent. A polar solvent is preferably used. Even more preferably, an ether solvent such as THF or methyl tert-butyl ether (MTBE) is used.

The amount of solvent used is generally between 0.1 and 20 ml per mmol of phosphonate.

The improvement in the selectivity of the reaction in the presence of the sequestering agent of the invention is observed irrespective of the temperature. The process of the invention may thus be performed at a temperature of 0° C. However, it is preferred to perform the process of the invention at a temperature of less than or equal to −20° C. and even more preferably at a temperature of less than or equal to −50° C.

As a guide, the reaction is generally performed at a temperature above −100° C.

Other aspects and advantages of the processes that are subjects of the invention will become apparent in the light of the examples given below as non-limiting illustrations.

### EXAMPLE 1

### Demonstration of the Effect of the Sequestering Agent TDA-1 and Comparison with 18-crown-6 at −78° C.

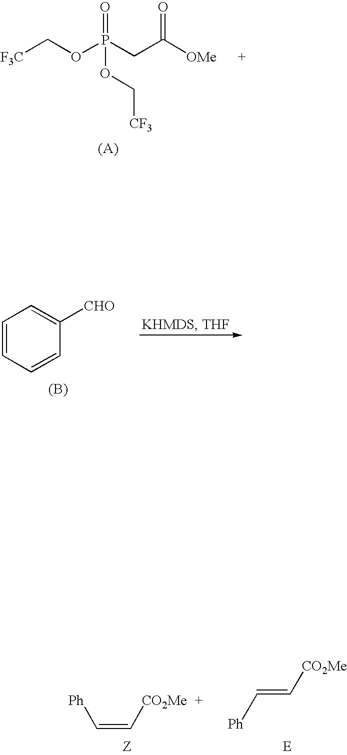

In this example, the phosphonate used corresponds to a phosphonate of formula (A) in which:

Y represents a radical CO₂R with R representing a methyl radical,

R₆ and R₇ represent a —CH₂CF₃ radical; and

R₈ represents a hydrogen atom.

The carbonyl compound (B) used is benzaldehyde.

The base used is potassium hexamethyldisilazide (KHMDS) as a 0.5M solution in toluene.

The solvent used is THF.

The sequestering agent of the invention used, known as TDA-1, corresponds to a tris(polyoxaalkyl)amine sequestering agent of formula (I) in which:

R₁, R₂, R₃ and R₄ represent a hydrogen atom;
n is 1;
R₅ represents a methyl radical.

Procedure:

1 mmol of phosphonate, 1.1 mmol of TDA-1 or 5 mmol of 18-crown-6 and 20 ml of anhydrous THF are introduced into a 100 ml one-necked flask. The mixture is then cooled using a bath of cardice and acetone. After stirring for thirty minutes at −78° C., 2 ml of a 0.5M solution of KHMDS in toluene are added dropwise. After stirring for a further thirty minutes, 1.1 mmol of benzaldehyde are added.

After about 2 hours at −78° C., the reaction is quenched by addition of saturated ammonium chloride solution and the mixture is extracted with toluene.

The mixture is analyzed by gas chromatography using a Varian Star 3400CX machine. The column used is a DB1 125-1034 from J&W Scientific (length: 30 m, inside diameter: 0.53 mm and film thickness of 3 μm). The initial column temperature is 100° C. and the temperature rise is 7° C. per minute. Under these conditions, the retention times of the various compounds are as follows:

benzaldehyde: 4.5 minutes

phosphonate: 5.9 minutes

Z isomer: 10.2 minutes

E isomer: 11.6 minutes

The diastereoselectivity factor S (S=Z/(Z+E) in %) is defined by the surface area ratio of the amount of Z isomer to the sum of the Z and E isomers formed.

The Z and E isomers are defined in the boxed reaction scheme on the previous page. In the present case, the role of the additive is to improve the selectivity of Z isomer.

The conversion (Conv=(Z+E)/(Z+E+phosphonate) in %) is also defined by the surface area ratio of the amount of olefin formed to the sum of the amounts of olefin formed and of residual phosphonate.

The diastereoselectivities obtained, without addition of sequestering agent, with addition of sequestering agent of the invention and with addition of 18-crown-6, at −78° C., are compared in Table I.

TABLE I

| Additive | Conv (%) | S (%) |
|---|---|---|
| none | 99 | 92 |
| 18-C-6 | 93 | 98 |
| TDA-1 | 94 | 98 |

The results obtained show the effect of the sequestering agent on the diastereoselectivity S expressed in %.

A diastereoselectivity of 98%, which is identical to that observed with 18-crown-6, is obtained with the sequestering agent of the invention known as TDA-1 at −78° C. The effect of TDA-1 is noted since the selectivity is only 92% without additive.

### EXAMPLE 2

### Evaluation of the Conditions of Example 1 (THF, KHMDS, TDA-1, −78° C.) with cyclohexanecarboxaldehyde

In this example, the procedure of example 1 is repeated and the nature of the aldehyde used is varied. The benzaldehyde is replaced with cyclohexanecarboxaldehyde (R₉ represents a cyclohexyl radical). The temperature is maintained at −78° C. for about 4 hours before allowing the system to return to room temperature overnight. The reaction medium is then worked up by addition of saturated ammonium chloride solution and extraction with toluene.

As in example 1, the mixture is analyzed by gas chromatography using a Varian Star 3400CX machine. The column used is a DB1 125-1034 from J&W Scientific (length: 30 m, inside diameter: 0.53 mm and film thickness of 3 μm). The initial column temperature is 100° C. and the temperature rise is 7° C. per minute. Under these conditions, the retention times (t_{R}) of the various compounds are as follows:

TABLE II

| Compound | t_{R} (min) |
|---|---|
| 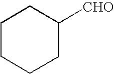 | 4.3 |
| 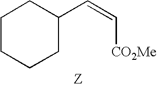 | 8.9 |
| 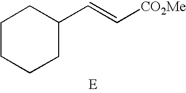 | 10.6 |

The selectivities obtained with TDA-1 and 1 8-crown-6 are indicated in Table III

TABLE III

Cyclohexanecarboxaldehyde

| Additive | Conv (%) | S (%) |
|---|---|---|
| 18-C-6 | 78 | 81 |
| TDA-1 | 100 | 81 |

It is observed that identical selectivities are obtained with TDA-1 and 18-crown-6.

### EXAMPLE 3

### Effect of Concentration Under the Conditions of Example 1 (THF, KHMDS TDA-1, −78° C., benzaldehyde)

The procedure of example 1 is repeated with TDA-1 and several tests are performed, reducing the amount of THF used.

The tests are performed with, respectively, 20 ml of THF (volume of example 1), 4 ml of THF, 2 ml of THF and without THF. This corresponds to phosphonate concentrations of 0.05M, 0.15M, 0.21M and 0.41M.

The results obtained are indicated in Table IV below.

TABLE IV

| C (M) | Conv (%) | S (%) |
|---|---|---|
| 0.05 | 97 | 98 |
| 0.15 | 100 | 98 |
| 0.21 | 100 | 98 |
| 0.41 | 76 | 77 |

The results obtained show that the concentration does not affect selectivity in the range 0.05-0.21M.

At 0.41M, the toluene of the KHMDS solution is the reaction solvent. The conversion is much slower and much less selective.

This shows the influence of the solvent on the diastereoselectivity of the reaction.

### EXAMPLE 4

### Use of Potassium Tert-Butoxide as Base Under the Conditions of Example 1 (THF, TDA-1, −78° C.)

Procedure:

1.05 mmol of KOTBu, 1.1 mmol of TDA-1 and 20 ml of anhydrous THF are introduced into a 100 ml one-necked flask. The solution is stirred for thirty minutes at room temperature. The mixture is then cooled using a bath of cardice and acetone. After stirring for thirty minutes at −78° C., 1 mmol of phosphonate is added dropwise. After stirring for a further thirty minutes, 1.1 mmol of aldehyde are added.

The temperature is maintained at −78° C. for about 4 hours, and the system is then allowed to return to room temperature overnight. The reaction medium is then worked up by addition of saturated ammonium chloride solution and extraction with toluene.

The results obtained are collated in the table below.

TABLE V

| Aldehyde | Conv (%) | S (%) |
|---|---|---|
| Benzaldehyde | 100 | 98 |
| Cyclohexanecarboxaldehyde | 100 | 82 |

The results obtained show that the selectivities towards Z isomers obtained with KOtBu are very similar to the selectivities obtained with KHMDS for the two aldehydes tested (cf. examples 1 and 2).

### EXAMPLE 5

### Effect of Concentration Under the Conditions of Example 4 (benzaldehyde, THF, KOtBu, TDA-1, −78° C.)

The procedure of example 4 is repeated with benzaldehyde, and several tests are performed, reducing the amount of THF used. The tests are performed with, respectively, 20 ml of THF (volume of example 4), 4 ml of THF, 2 ml of THF and 1 ml of THF. This corresponds to phosphonate concentrations of 0.05M, 0.21M, 0.37M and 0.60M.

The results obtained are indicated in Table VI below.

TABLE VI

| C (M) | Conv (%) | S (%) |
|---|---|---|
| 0.05 | 84 | 98 |
| 0.21 | 91 | 96 |
| 0.37 | 99 | 94 |
| 0.60 | 99 | 94 |

### EXAMPLE 6

General procedure: 5.5 mmol of phosphonate identical to the phosphonate of example 1, 5.5 mmol of TDA-1 and 90 ml of anhydrous THF are introduced into a 250 ml round-bottomed flask. The mixture is then cooled using a bath of cardice and acetone. After stirring at −78° C. for 30 minutes, 10.5 ml of a 0.5M solution of KHMDS in toluene are added. After stirring for a further 30 minutes, 5 mmol of aldehyde are added and the medium is stirred at −78° C.

As regards entries 1, 2, 3, 4 and 7, the medium is worked up after reaction at −78° C. for 4 hours. For entries 5, 6 and 8, since the aldehydes are less reactive, the medium is allowed to return to room temperature overnight before work-up.

Work-up: The mixture is diluted with 70 ml of methyl tert-butyl ether (MTBE) and quenched with 50 ml of saturated aqueous NH₄Cl solution. The aqueous phase is re-extracted twice with 20 ml of MTBE and the combined organic phases are washed until neutral. After drying over Na₂SO₄, the solvent is evaporated off under vacuum and the mixture of Z+E olefins is purified by flash chromatography (mixture of cyclohexane and ethyl acetate). The yields indicated in the table below are thus isolated yields. The selectivity is determined by integration on the vinyl protons in proton NMR and in accordance with measurements taken by gas chromatography.

TABLE VII

| Entry | Aldehyde | S (%) | Yield (%) |
|---|---|---|---|
| 1 | 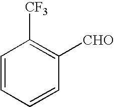 | 99 | 95 |
| 2 | 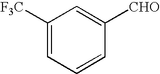 | 98 | 90 |
| 3 | 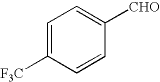 | 99 | 91 |
| 4 | 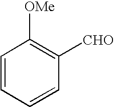 | 98 | 95 |
| 5 | 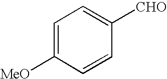 | 93 | 92 |
| 6 | 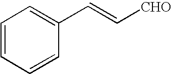 | 93 | 95 |
| 7 | 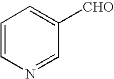 | 98 | 93 |
| 8 | 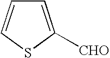 | 92 | 97 |

1-21. (canceled)

22- A process for the diastereoselective preparation of olefins (C) via the Homer-Wadsworth-Emmons reaction, comprising the step of reacting at low temperature a phosphonate (A) with a carbonyl derivative (B) in the presence of a base, in a suitable solvent to form a reaction medium, 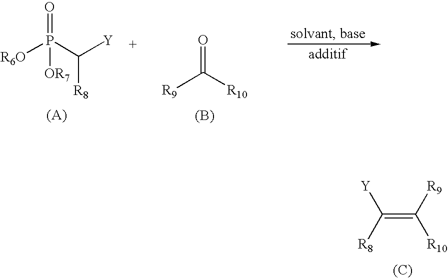
in which the compounds (A) (B) and (C) are such that:
Y represents an electron-withdrawing group of the formula:
—CO₂R,
—CN,
—C(O)R,
—S(O)R,
—S(O)₂R,
—C(O)NRR′,
—N═CRR′, or
—P(O)OROR′,
with R and R′ as defined below,
R₆ and R₇, taken independently, are identical or different and represent:
a saturated or unsaturated, linear or branched aliphatic radical containing from 1 to 24 carbon atoms, optionally substituted with hetero atoms;
a saturated, unsaturated or aromatic, monocyclic or polycyclic cycloaliphatic radical containing from 4 to 24 carbon atoms, optionally substituted with hetero atoms; or
a saturated or unsaturated, linear or branched aliphatic radical bearing a cyclic substituent optionally substituted with hetero atoms in the aliphatic part and/or the cyclic part;
R₁₀, R and R′, taken independently, are identical or different and represent:
a hydrogen atom;
a saturated or unsaturated, linear or branched aliphatic radical having from 1 to 24 carbon atoms, optionally substituted with hetero atoms;
a saturated, unsaturated or aromatic, monocyclic or polycyclic cycloaliphatic radical having from 4 to 24 carbon atoms, optionally substituted with hetero atoms; or
a saturated or unsaturated, linear or branched aliphatic radical bearing a cyclic substituent optionally substituted with hetero atoms in the aliphatic part and/or the cyclic part;
R₆, R₇, R and R′ taken together optionally form a saturated, unsaturated or aromatic ring optionally comprising hetero atoms;
R₈ represents a radical of the formula:
—R,
a halogen atom,
—OR, or
—NRR′,
with R and R′ as defined above,
R₉ represents a radical of the formula:
a saturated or unsaturated, linear or branched aliphatic radical having from 1 to 24 carbon atoms, optionally substituted with hetero atoms;
a saturated, unsaturated or aromatic, monocyclic or polycyclic cycloaliphatic radical having from 4 to 24 carbon atoms, optionally substituted with hetero atoms; the hetero atoms also possibly being present in the cyclic part; or
a saturated or unsaturated, linear or branched aliphatic radical bearing a cyclic substituent optionally substituted with hetero atoms in the aliphatic part and/or the cyclic part;
with the further proviso that R₉ takes precedence over R₁₀ according to the Cahn Ingold and Prelog rules, and
wherein a tris(polyoxaalkyl)amine sequestering agent of formula (I):
N—[CHR₁—CHR₂—O—(CHR₃—CHR₄—O)ₙ—R₅]₃ (I)
in which:
n is an integer between 0 and 10;
R₁, R₂, R₃ and R₄ are identical or different and represent a hydrogen atom or an alkyl radical having from 1 to 4 carbon atoms; and
R₅ represents a hydrogen atom, an alkyl or cycloalkyl radical containing up to 12 carbon atoms, a phenyl radical or a radical of formula —CₘH₂ₘ-Φ, or CₘH₂ₘ₊₁-Φ-, with m being an integer between 1 and 12 and Φ being a phenyl radical;
is being added to the reaction medium in an amount that is effective to increase the diastereoselectivity of the olefins (C).

23- The process as claimed in claim 22, wherein the tris(polyoxaalkyl)amine sequestering agent is a tris(polyoxaalkyl)amines of formula (I) wherein:
R₁, R₂, R₃ and R₄ represent a hydrogen atom or a methyl radical;
n is an integer between 0 and 3; and
R₅ represents a hydrogen atom or an alkyl radical having from 1 to 4 carbon atoms.

24- The process as claimed in claim 23, wherein in formula (I):
R₁, R₂, R₃ and R₄ represent a hydrogen atom;
n is 1; and
R₅ represents a methyl radical.

25- The process as claimed in claim 22, wherein the tris(oxaalkyl)amine sequestering agent of formula (I) is used in an amount of between 0.05 and 10 equivalents per 1 equivalent of phosphonate, one equivalent of aldehyde and one equivalent of base.

26- The process as claimed in claim 25, wherein the amount of tris(oxaalkyl)amine sequestering agent of formula (I) used is I equivalent of tris-(oxaalkyl)amine sequestering agent of formula (I) per 1 equivalent of phosphonate, one equivalent of aldehyde and one equivalent of base, the whole being dissolve in the solvent.

27- The process as claimed in claim 22, wherein in formula (A):
Y represents CO₂R, with R representing a hydrogen atom or a linear, branched or cyclic, saturated or unsaturated alkyl radical alkyl having from 1 to 12 carbon atoms,
R₆ and R₇ represent a —CH₂CF₃ radical, and
R₈ represents a hydrogen atom.

28- The process as claimed in claim 22, wherein in formula (A):
Y represents CO₂R, with R representing a methyl radical,
R₆ and R₇ represent a —CH₂CF₃ radical, and
R₈ represents a hydrogen atom.

29- The process as claimed in claim 22, wherein the carbonyl derivative is an aldehyde, with R₁₀ representing a hydrogen atom.

30- The process as claimed in claim 29, wherein R₉ is an aliphatic radical, optionally having ethylenic unsaturations.

31- The process as claimed in claim 9, wherein the radical R₉ is cyclohexyl.

32- The process as claimed in claim 9, wherein R₉ is aromatic, optionally having one or more substitutions with alkoxy groups having from 1 to 6 carbon atoms, halogen atoms or CF₃ groups.

33- The process as claimed in claim 32, wherein the radical R₉ is a phenyl radical.

34- The process as claimed in claim 22, wherein the base used is:
an amide of the formula: MNR″R′″ with M being an alkali metal, and R″, R′″ being alkyl or alkylsilane radicals,
an alkoxide of the formula: MOR″ with M being an alkali metal and R″ being an alkyl radical,
an hydride of the formula: MH with M being an alkali metal,
a carbonate of the formula: M₂CO₃, with M being an alkali metal or an alkaline-earth metal,
an alkali metal or alkaline-earth metal hydroxide, or
an organic base, in combination with alkali metal or alkaline-earth metal halides.

35- The process as claimed in claim 34, wherein the base is the potassium salt of hexamethyldisilazane (KHMDS) and potassium tert-butoxide (KOtBu).

36- The process as claimed in claim 22, wherein the solvent is a polar solvent.

37- The process as claimed in claim 36, wherein the solvent used is an ether solvent.

38- The process as claimed in claim 37, wherein the solvent used is tetrahydrofuran (THF) or methyl tert-butyl ether (MTBE).

39- The process as claimed in claim 36, wherein the solvent is used in an amount of between 0.1 and 20 ml per mmol of phosphonate (A).

40- The process as claimed in claim 22, wherein the reaction medium is maintained at a temperature of less than or equal to 0° C.

41- The process as claimed in claim 40, wherein the temperature is maintained at a temperature of less than or equal to −20° C.

42- The process as claimed in claim 41, wherein the temperature is maintained at a temperature of less than or equal to −50° C.
